(19) United States
(12) Patent Application Publication
(10) Pub. No.: US 2012/0172911 A1
(43) Pub. Date: Jul. 5, 2012

(54) OCCLUSION DEVICE

(75) Inventor: Jonathan Brister Welch, Greencastle, IN (US)

(73) Assignee: Cook Medical Technologies LLC, Bloomington, IN (US)

(21) Appl. No.: 13/338,749

(22) Filed: Dec. 28, 2011

Related U.S. Application Data
(60) Provisional application No. 61/428,428, filed on Dec. 30, 2010.

(51) Int. Cl. A61M 29/00

(52) U.S. Cl. 606/194

(57) ABSTRACT

A vascular occlusion device for occluding a body cavity includes an elongate member with a first lumen and a second lumen. An inflatable balloon is disposed about a distal end of the elongate member, and is inflated with inflating fluid introduced into the interior of the balloon by way of the first lumen. The device also includes a pressure regulation system that determines the pressure of embolization material being injected from the second lumen into the body cavity to occlude the body cavity.

### CROSS-REFERENCE TO RELATED APPLICATIONS

This application claims the benefit of U.S. Provisional Application No. 61/428,428, filed on Dec. 30, 2011, entitled “OCCLUSION DEVICE,” the entire contents of which are incorporated herein by reference.

### BACKGROUND

The present invention generally relates to vascular occlusion devices.

A number of different devices may be used to occlude a body cavity including, for example, a blood vessel. An example of an occlusion device includes embolization coils. Embolization coils are permanent and promote blood clots or tissue growth over a period of time, thereby occluding the body cavity. However, while the blood clots or the tissue grows, blood may continue to flow past the coil and through the body cavity. It may take a significant period of time for sufficient tissue to grow to fully occlude the body cavity. This leaves a patient open to a risk of injury from the condition which requires the body cavity be occluded. An example of such a condition includes, but is not limited to, an atrial septal defect such as a patent foramen ovale. When it is desirable to quickly occlude a blood vessel, an inflatable balloon may be used, and embolization material may be injected into the vessel.

### BRIEF SUMMARY

In one form, a vascular occlusion device for occluding a body cavity includes an elongate member with an inflation lumen or a first lumen and an occlusion lumen or a second lumen. An inflatable balloon is disposed about a distal end of the elongate member, and is inflated with inflating fluid introduced into the interior of the balloon by way of the first lumen. The device also includes a pressure regulation system that determines the pressure of embolization material being injected from the second lumen into the body cavity to occlude the body cavity.

Further features and advantages of the invention will become readily apparent from the following description and from the claims.

### BRIEF DESCRIPTION OF THE DRAWINGS

FIG. 1a is an exploded view of an embolization kit with a vascular occlusion device in accordance with an embodiment of the present invention;

FIG. 1b is a side view of an embolization kit with a vascular occlusion device in accordance with an embodiment of the present invention;

FIG. 2 is a sectional view through the distal end of the vascular occlusion device of FIG. 1b along the line 2-2;

FIG. 3 is a cross-sectional environmental view of the vascular occluding device of FIGS. 1a and 1b in a body vessel; and

FIG. 4 is a flowchart illustrating a method of delivering the vascular occlusion device of FIGS. 1a and 1b.

### DETAILED DESCRIPTION

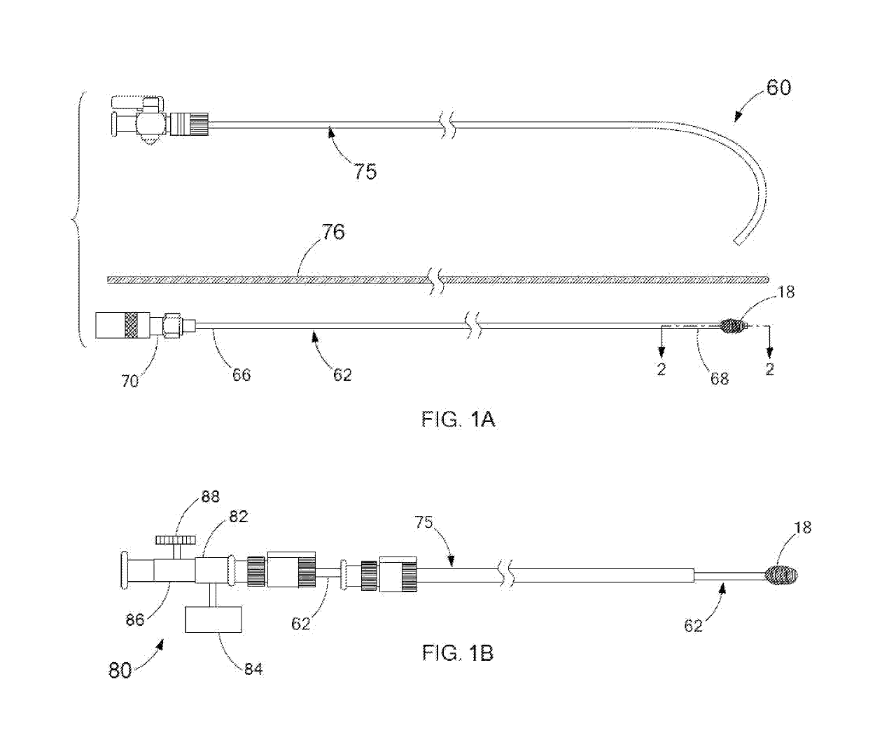

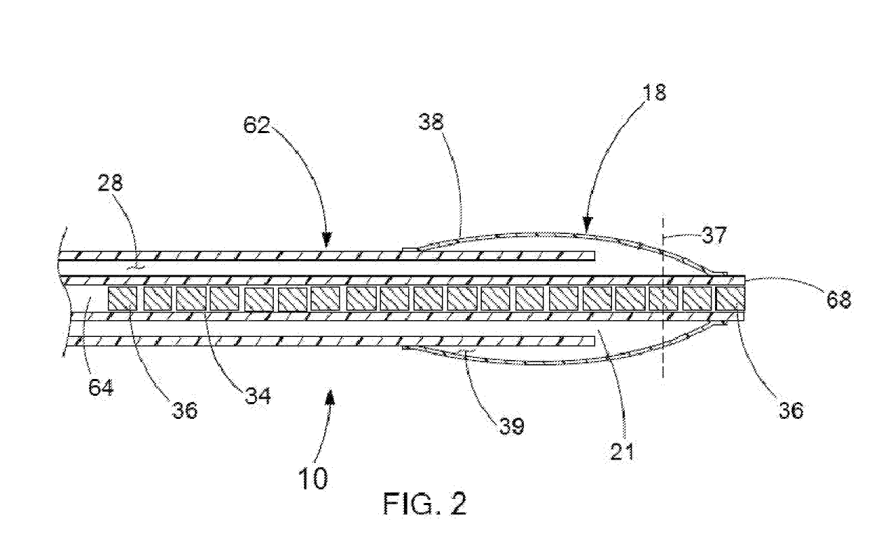

Referring now to FIGS. 1a, 1b, and 2, an embolization kit embodying the principles of the present invention is illustrated therein and designated at 60. As its primary components, the system 10. As shown, the kit 60 includes a vascular occlusion device 62 such as microcatheter defining a catheter lumen 64 and is preferably made of a soft, flexible material such as silicone or any other suitable material. Generally, the vascular occlusion device 62 has a proximal end 66, and a distal end 68, and a plastic adapter or hub 70. In one example, the hub 70 receives the embolization particles 36 to be advanced therethrough, such as for example, in a mixture or slurry with a fluid, e.g., saline solution, as will be discussed in further detail below. In another example, the embolization particles 36 may be pre-packaged within the lumen 64 of the vascular occlusion device 62 and advanced therethrough by advancing a fluid, e.g., saline solution, contrast media, a mixture thereof or the alike, through the hub 70 as will also be discussed in further detail below.

The kit 60 may further include a guide catheter 75 and a guide wire 76 which provides the guide catheter 75 a path during insertion of the guide catheter 75 within a body vessel. The size of the guide wire 76 is based on the inside diameter of the guide catheter 75.

In one embodiment, the guide catheter 75 is a polytetrafluoroethylene (PTFE) guide catheter or sheath for percutaneously introducing the vascular occlusion device 62 into a body vessel. Of course, any suitable material may be used without falling beyond the scope or spirit of the present invention. The guide catheter 75 may have a size of about 4-8 french and allows the vascular occlusion device 62 to be inserted therethrough to a desired location in the body vessel. The guide catheter 75 receives the vascular occlusion device 62 and provides stability of the vascular occlusion device 62 at a desired location within the body vessel. For example, the guide catheter 75 may stay stationary within a common visceral artery, e.g., a common hepatic artery, adding stability to the vascular occlusion device 62 as the vascular occlusion device 62 is advanced through the guide catheter 75 to a point of occlusion in a connecting artery, e.g., the left or right hepatic artery.

When the distal end 68 of the vascular occlusion device 62 is at a point of occlusion in the body vessel, the embolization particles 36 may be loaded at the proximal end 66 via the hub 70 of the vascular occlusion device 62. In one example, saline solution is mixed with the embolization particles to form a slurry which is injected into the hub 70 of the vascular occlusion device 62 and advanced through the lumen 64. Alternatively and as illustrated in FIG. 2, the embolization particles 36 may be pre-loaded within the lumen 64 of the vascular occlusion device 62. In this example, the lumen 64 has a cross-section 37 that corresponds to the cross-sections of the particles 36 so as to position the particles 36 such that their longitudinal axes are aligned with each other and with a central longitudinal axis of the lumen 64. Saline solution or other suitable transferring fluid is introduced at the proximal end 66 of the vascular occlusion device 62 to advance the embolization particles 36 to the distal end 68 of a lumen 64. Providing the embolization particles 36 with such a consistent orientation facilitates or insures deeper penetration of the particles 36 into the body vessel, such as for example, into a tumor vascular bed. Alternatively, a push wire (not shown) may be used to mechanically advance or push the embolization particles 36 through the vascular occlusion device 62. The size of the push wire depends on the diameter of the vascular occlusion device 62.

It is to be understood that the body vessel embolization kit described above is merely one example of a kit that may be used to deploy the embolization particles into the body vessel. Of course, other kits, assemblies, and systems may be used to deploy any embodiment of the embolization particles without falling beyond the scope or spirit of the present invention, such as for example, a vascular occlusion device having two lumens; one lumen for advancing the embolization particles, and the second lumen for being advanced along the guide wire to a desired point of occlusion.

Notably, the present invention as discussed in the foregoing paragraphs provides at least two means for delivering a medicant to a targeted body vessel site. Specifically, the medicant may be delivered either as incorporated into the biocompatible material of the embolization particles 36 or as a coating on the embolization particles 36. Preferably, the local delivery of the medicant includes minimizing the side effects to the healthy tissues which may otherwise be an issue if delivered systematically to treat certain illnesses or conditions.

Note that “Embolization particle” is a generic term for a particle used to artificially block blood flow. Embolization of a vessel to an organ or in an organ may be used for a number of reasons. Vessel embolization may be used, for instance, for 1) controlling a bleeding caused by trauma, 2) prevention of profuse blood loss during an operation requiring dissection of blood vessels, 3) obliteration of a portion of or of a whole organ having a tumor, or 4) blocking of blood flow into normal blood vessel structures such as AVM's and aneurysms.

The embolization particles 36 may be formed from a biocompatible material. The biocompatible material may be a non-biodegradable material, such as for example, glass (E-glass, S-glass or otherwise) or a non-biodegradable polymer, e.g., PTFE. Alternatively, the biocompatible material may be a biodegradable polymer, such as for example, polylactic acid (PLA), poly(glycolic acid) (PGA), copolymers of the PLA and PGA, or polycaprolactone (PCL). These synthetic biopolymers exhibit good mechanical properties. Moreover, the degradation products, such as glycolic acid for PGA, are also non-toxic and easily metabolized by the body.

The biocompatible material may include various types of additives. In one embodiment, the biocompatible material contains a radiopacifier. The radiopacifier is detectable within the body of a patient by fluoroscopic visualization and/or X-ray and thus, allows an interventionalist to monitor its location when positioned within a patient's body.

In another embodiment, the biocompatible material contains a medicant additive. The medicant may be homogenously dispersed throughout the biocompatible material or alternatively, be positioned in discrete areas or regions within or about the biocompatible material, e.g., forming either an outer, intermediate or inner layer with the biocompatible material for example via a co-extrusion process or the alike. The medicant may include but is not limited to a compound or compounds to promote blood clotting, an antiangiogenic which inhibits the growth of new blood vessels, or a cytotoxic drug used to stop the proliferation of cancer cells. For instance, the biocompatible material may be a synthetic biopolymer which has trapped chemotherapeutic agents within. Inside the body of the patient, the polymer degrades and the chemotherapeutic agents can diffuse into the immediately adjacent tissue. The rate of degradation of the biopolymer may be tailored to control the diffusion of the chemotherapeutic agent (or other medicant) for a specific medical application and accordingly, may be rapid, slow or anywhere therebetween.

In at least one embodiment, the embolization particles 36 are coated with a medicant (notably, in other embodiments the embolization particles 36 may be without a medicant 38 coating). The medicant coating may be sprayed via a coating spray device. The thickness of the coating may be relatively thin, such as for example, on the order of several angstroms, however, thicker coatings may be used without departing from the present invention.

Further details of embolization particles may be found in U.S. patent application Ser. No. 12/193,368, filed Aug. 18, 2008, the entire contents of which are incorporated herein.

In some arrangements, the embolization material may include any appropriate biocompatible material having an appropriate viscosity allowing it to flow through the second lumen 64 into the body cavity. In some examples, the occlusive material may be an appropriate adhesive for permanently bonding to body tissue to occlude the body cavity. In other examples, the embolization material may be configured to promote body tissue growth to occlude the body cavity. Some examples of an adhesive include, but are not limited to, polyvinyl alcohol (PVA) and cyanoacrylate adhesives. An example of a material to promote body tissue growth includes, but is not limited to, extra cellular matrix (ECM). In other examples, it may be possible to use a combination of an adhesive and the extra cellular matrix to occlude the body cavity.

As known, ECM is a complex structural entity surrounding and supporting cells found within tissues. More specifically, ECM includes structural proteins (for example, collagen and elastin), specialized protein (for example, fibrillin, fibronectin, and laminin), and proteoglycans, a protein core to which are attached long chains of repeating disaccharide units termed glycosaminoglycans.

In a preferred embodiment, the extracellular matrix is comprised of small intestinal submucosa (SIS). As known, SIS is a resorbable, acellular, naturally occurring tissue matrix composed of ECM proteins and various growth factors. SIS is derived from the porcine jejunum and functions as a remodeling bioscaffold for tissue repair. SIS has characteristics of an ideal tissue engineered biomaterial and can act as a bioscaffold for remodeling of many body tissues including skin, body wall, musculoskeletal structure, urinary bladder, and also supports new blood vessel growth. SIS may be used to induce site-specific remodeling of both organs and tissues depending on the site of implantation. In practice, host cells are stimulated to proliferate and differentiate into site-specific connective tissue structures, which have been shown to completely replace the SIS material in time.

In this embodiment, SIS may be provided in a fluid form including, for example, a gel. The gel SIS may be used to adhere to walls of the body cavity in which the occlusion device 62 is deployed and to promote body tissue growth within the body cavity. SIS has a natural adherence or wetability to body fluids and connective cells comprising the connective tissue of the walls of a body cavity. Since the embolization material provided by the occlusion device 62 is intended to permanently occlude the body cavity, the distal end 68 is positioned such that the SIS may be introduced into contact with host cells of the wall such that the walls will adhere to the SIS and subsequently differentiate, growing into the SIS and eventually occluding the body cavity with the tissue of the walls to which the substance was originally introduced.

As shown in FIGS. 1a, 1b, and 2, an inflatable proximal balloon 18 is disposed about the distal end 68 of the vascular occlusion device 62. An inflation lumen or a first lumen 28 is longitudinally formed in the vascular occlusion device 62. In a preferred embodiment, an inner wall 34 defines the inner lumen 64 longitudinally extending through the vascular occlusion device 62.

As shown, the balloon 18 has a balloon wall 38 disposed about the circumference of the distal end 68 and defines a balloon interior 39. An inflation orifice 21 and the distal end of the first lumen 28 is configured to introduce an inflation fluid provided from, for example, the proximal end 66 of the vascular occlusion device 62 through the first lumen 28, into the balloon interior 39 to inflate and expand the balloon 18. The inflation fluid may include any appropriate biocompatible fluid for inflating the balloon 18 and later deflating of the balloon.

Further details may be found in U.S. patent application Ser. No. 11/848,777, filed Aug. 31, 2007, the entire contents of which are incorporated herein.

In some implementations, as shown in FIGS. 1A and 1B, the embolization kit 60 includes a pressure regulation system 80 connected to the proximal end of the occlusion device 62. The pressure regulation system 80 includes a pressure sensor 82 that sends signals to a pressure monitor 84, which displays the pressure of the occlusion device 62. The system 80 further includes a pressure valve 86 to control the pressure in the body vessel in which the occlusion device 62 is inserted as the occlusion particles 36 or occlusion fluid is injected into the vessel 100.

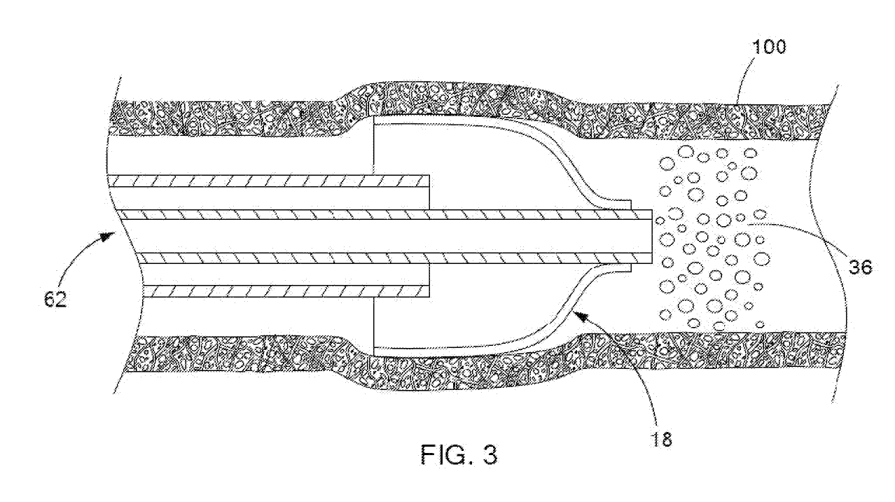

When the embolization kit 60 is in use, as shown in FIG. 3, the embolization particles or solution are injected into a body vessel 100, or a body cavity. As the embolizaiton material is injected into the vessel 100, the pressure sensor 82 evaluates the pressure in the body vessel 100 and sends information regarding the pressure to the monitor 84 which displays the pressure to an operator of the kit 60 such as, for example, a clinician. When the pressure in the body vessel 100 exceeds a predetermined threshold, the valve 86 terminates any additional embolization material from being injected into the body vessel 100. The clinician, however, is able to override the valve 86 with an override switch or knob 88 if the clinician wishes to inject additional embolization material into the body vessel 100.

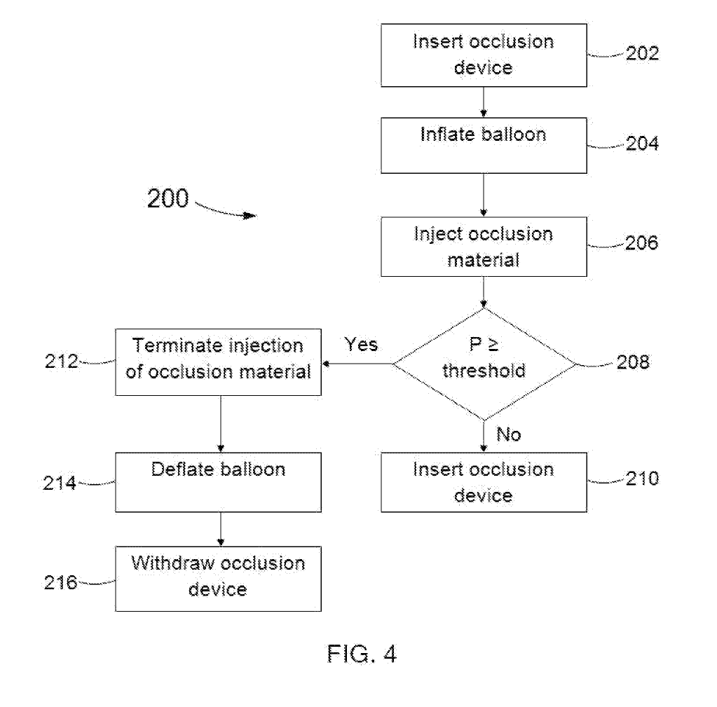

Referring to FIG. 4, there is shown a process 200 that implements the embolization kit 60. In step 202, the occlusion device 62 is inserted in a body vessel, and in step 204, the balloon 18 is inflated after the distal end of the occlusion device positioned at the desired location in the body vessel. In step 206, emobolization material is injected into the body vessel. In decision step 208, the system 80 determines if the pressure (P) in the body vessel exceeds a predetermined threshold pressure. If the pressure is below the threshold, then the injection of embolization material continues (step 210). If the pressure is equal to or greater than the threshold, then the injection of the embolization material terminates. As mentioned above, the clinician may adjust the override knob 88 to inject additional embolization material into the body vessel. After the desired amount of embolization material is injected into the body vessel, the clinician, in step 214, deflates the balloon 18 and withdraws the occlusion device 62 (step 216).

It is understood that the assembly described above is merely one example of an assembly that may be used to deploy the occlusion device in a body vessel. Of course, other apparatus, assemblies and systems may be used to deploy any embodiment of the occlusion device without falling beyond the scope of the following claims.

1. A vascular occlusion device for occluding a body cavity comprising:
an elongate member extending from a proximal end to a distal end, a first lumen and a second lumen being formed longitudinally in the elongate member;
an inflatable balloon including a balloon wall disposed about the distal end of the elongate member, the balloon wall defining a balloon interior, inflation fluid being introduced into the balloon interior by way of the first lumen;
embolization material to be injected from the second lumen into the body cavity to occlude the body cavity; and
a pressure regulation system that determines the pressure of embolization material being injected from the second lumen into the body cavity to occlude the body cavity.

2. The occlusion device of claim 1 wherein the pressure regulation system includes a pressure sensor that evaluates the pressure.

3. The occlusion device of claim 2 wherein pressure regulation system includes a pressure monitor that displays pressure to an operator of the device.

4. The occlusion device of claim 1 wherein in the pressure regulation system includes pressure valve that terminates the injection of the embolization material when the pressure is equal to or exceeds a predetermined threshold pressure.

5. The occlusion device of claim 4 wherein the pressure regulation system includes an override mechanism to enable injecting embolization material when the pressure exceeds the threshold pressure.

6. The occlusion device of claim 1 wherein the embolization material is an adhesive.

7. The occlusion device of claim 6 wherein the embolization material includes at least one of a polyvinyl alcohol and cyanoacrylate.

8. The occlusion device of claim 1 wherein the embolization material promotes body tissue growth.

9. The occlusion device of claim 8 wherein the embolization material is an extracellular matrix.

10. The occlusion device of claim 9 wherein the extracellular matrix includes small intestinal submucosa.

11. The occlusion device of claim 1 wherein the embolization material includes embolization particles.

12. The occlusion device of claim 1 wherein the embolization particles are coated with a medicant.

13. A method of occluding a body cavity comprising:
positioning a distal end of an occlusion device at or near a desired treatment area;
inflating a balloon located at the distal end of the occlusion device such that the balloon contacts the interior wall of the body cavity;
injecting embolization material into the body cavity; and
regulating the pressure of the embolization material.

14. The method of claim 13 wherein regulating the pressure includes determining the pressure with a pressure sensor.

15. The method of claim 13 wherein regulating the pressure includes monitoring the pressure.

16. The method of claim 13 wherein regulating the pressure includes terminating the injection of embolization material when the pressure is equal to or exceeds a threshold pressure.

17. The method of claim 16 wherein regulating the pressure includes overriding the termination of the injection of embolization material to continue injecting embolization material into the body cavity.

18. The method of claim 13 wherein the embolization material is a fluid.

19. The method of claim 13 wherein the embolization material includes embolization particles.

20. The method of claim 13 wherein the embolization material includes at least one of a polyvinyl alcohol, cyanoacrylate, and small intestine submucosa.
